(19) United States
(12) Patent Application Publication
(10) Pub. No.: US 2003/0228627 A1
(43) Pub. Date: Dec. 11, 2003

(54) Assay for p53 function in cells

(76) Inventors: Beverly M. Emerson, San Diego, CA (US); Joaquin Espinosa, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/397,131

(22) Filed: Mar. 24, 2003

Related U.S. Application Data
(60) Provisional application No. 60/366,897, filed on Mar. 22, 2002.

(51) Int. Cl. G01N 33/574

(52) U.S. Cl. 435/7.1; 435/7.23

(57) ABSTRACT

Disclosed herein is an assay for identifying compounds that modulate the interaction between p53 and specific DNA binding sites. The assay uses chromatin-assembled target genes to correctly reproduce p53 binding to target genes of interest. This assay permits the selection of compounds which preferentially associate with p53 when bound to specific DNA binding sites but not others. Moreover, compounds can be screened for their ability to selectively modulate p53 interaction with specific binding sites associated with individual genes. Compounds can also be screened for their ability to selectively modulate p53 interaction with specific cofactors (proteins, nucleic acids, peptides, etc.) when p53 is bound to specific DNA sequences. This system allows drugs to be tailored to affect p53-dependent regulation of certain target genes that regulate distinct cellular pathways without affecting other genes and pathways.

### RELATED APPLICATIONS

This applications claims the benefit of priority under 35 U.S.C. 119(e) of U.S. Provisional Application No. 60/366,897, filed Mar. 22, 2002.

### GOVERNMENTAL INTEREST

This invention was made with support from Grant No. 2PO1-CA-54418-11 from the National Institutes of Health. The government may have rights in this invention.

### FIELD OF THE INVENTION

The present invention relates to new assays for p53 function in cells. In particular, this invention relates to assays for compounds that affect binding of p53 to chromatic-assembled target genes.

### BACKGROUND OF THE INVENTION

The p53 tumor suppressor gene is the most frequent target for genetic alterations in cancer, with mutations occurring in approximately 50% of all human tumors. The importance of p53 in cancer prevention results from its ability to regulate such critical processes as cell cycle progression and apoptosis (Levine, A. J. 1997 Cell 88:323-331; Prives, C. and Hall, P. A. 1999 J Pathol 187:112-126; Vogelstein, B. et al. 2000 Nature 408:307-310). p53 is a sequence-specific DNA binding protein that has been shown to activate transcription of a number of target genes, including p21, Mdm2, GADD45, Bax, and cyclin G. Induction of p21 results in cell cycle arrest in response to DNA damage by inhibiting cyclin-dependent kinase activity (El-Deiry, W. S. et al. 1993 Cell 75:817-825; Xiong, Y. et al. 1993 Nature 366:701-704). p21 gene is a natural and direct target of p53 and is regulated in an inducible (El-Deiry, W. S. et al. 1993 Cell 75:817-825) and a constitutive basal level manner (Tang, H. Y. et al. 1998 J Biol Chem 273:29156-29163) through two consensus p53 promoter binding sites at −2.3 kb (5′) and −1.4 kb (3′).

The p53 activation domain interacts with transcriptional coactivators, CREB binding protein (CBP) and p300 (CBP/p300), and both CBP/p300 and p300/CBP-associated factor (PCAF) increase the ability of p53 to activate p21 gene expression in vivo (Scolnick, D. M. et al. 1997 Cancer Res 57:3693-3696). Activation of Bax and other genes by p53 promotes apoptosis (Miyashita, T. and Reed, J. C. 1995 Cell 80:293-299). The regulation of cell cycle arrest and apoptosis by p53 are mechanistically distinct processes (Attardi, L. D. et al. 1996 EMBO J 15:3693-3701). Recently, oligonucleotide microarray analysis has identified a broad spectrum of genes that are controlled by p53 in a positive or negative manner, and whose functions fall into categories that reflect the role of p53 as an integrator of diverse cellular signals (Zhao, R. et al. 2000 Genes & Dev 14:981-993).

The 393 amino acid p53 protein consists of two N-terminal activation domains (amino acids 142; 42-83), a sequence-specific DNA binding domain (amino acids 102-292), and a C-terminal oligomerization domain (amino acids 324-355). The DNA binding activity of p53 is thought to be under negative constitutive regulation through two regions within its inhibitory C-terminal domain (amino acids 290-325 and 356-393) (Hupp, T. R. et al. 1992 Cell 71:875-886). This inhibition can be relieved by acetylation, phosphorylation, or protease cleavage.

After DNA damage, p53 is phosphorylated and acetylated at a number of sites within its N- and C-termini. Phosphorylation within the N-terminal activation domain most likely affects its interactions with Mdm2, which controls p53 stability, and components of the transcription initiation machinery (Prives, C. 1998 Cell 95:5-8). Acetylation of p53 by the histone acetyl transferases (HAT) CBP/p300 and PCAF activates DNA binding in vitro and each HAT can coactivate p53-dependent transcription in transient expression experiments (Gu, W. and Roeder, R. G. 1997 Cell 90:595-606; Avantaggiati, M. L. et al. 1997 Cell 89:1175-1184; Lill, N. L. et al. 1997 Nature 387:823-827; Scolnick, D. M. et al. 1997 Cancer Res 57:3693-3696; Liu, L. et al. 1999 Mol Cell Biol 19:1202-1209).

### SUMMARY OF THE INVENTION

One embodiment of the invention is an assay which allows the identification of compounds that modulate the interaction between p53 and specific DNA binding sites using chromatin-assembled p53 target genes. Thus, in one embodiment, test compounds are contacted with p53 and chromatin-assembled p53 target genes in order to determine whether the compounds enhance, or reduce the level of p53 binding to the target gene.

One embodiment of the invention is a method for identifying compounds that modulate the binding of p53 protein to a gene of interest that includes providing the gene of interest as chromatin-assembled DNA; contacting the gene of interest with the p53 protein and a test compound; and determining whether the presence of the test compound modulates the binding of the p53 to the gene of interest.

Another embodiment is a method for identifying a modulator that inhibits growth of cancer cells that includes providing a gene construct comprising a chromatin-assembled promoter linked to a reporter gene, wherein the promoter binds p53 protein; incubating the gene construct in the presence of p53 protein and a test compound; and determining whether the test compound increases binding of the p53 protein to the gene construct by measuring the amount of reporter gene activity in the presence and the absence of the test compound.

Still another embodiment is a method for identifying a test compound that modulates the interaction of p53 protein with a transcription cofactor that associates with p53 protein when p53 protein is bound to a gene of interest that includes providing the gene of interest as chromatin-assembled DNA; contacting the gene of interest with the p53 protein, the transcription cofactor, and the test compound; and determining whether the presence of the test compound modulates the association of the cofactor with the p53 protein bound to the gene.

One other embodiment is a kit for identifying test compounds that affect p53 protein binding to a promoter. The kit includes a gene construct comprising a chromatin assembled p53 binding site genetically linked to a reporter gene; and isolated p53 protein.

### BRIEF DESCRIPTION OF THE DRAWINGS

FIG. 1(A) is a schematic diagram of the p21 promoter. Relative positions to the transcription initiation site and sequences of the p53 5′ and 3′ binding sites are indicated. The p53 3′ binding site preferably includes the nucleotides 5′-AGACTGGGCATGTCTGGGCA-3′ (SEQ ID NO: 1) and its opposite strand 3′-TCTGACCCGTACAGACCCGT-5′ (SEQ ID NO: 2). The p53 5′ binding site preferably includes the nucleotides 5′-GAACATGTCCCAACATGTTG-3′ (SEQ ID NO: 3) and its opposite strand 3′-CTTGTACAGGGTTGTACAAC-5′ (SEQ ID NO: 4).

FIG. 1(B) is a schematic representation of one embodiment of an in vitro chromatin assembly and transcription protocol.

### DETAILED DESCRIPTION OF THE PREFERRED EMBODIMENT

Embodiments of the invention concern in vitro test methods and kits to determine p53 binding and transcriptional activation using DNA or chromatin-assembled target DNA. In one embodiment, the invention relates to assays to discover modulators that affect p53 binding or function (hereinafter “p53 modulators”), by screening those compounds for their ability to interact with a promoter on a chromatin-assembled target gene, such as p21, Mdm2, GADD45, Bax, or cyclin G. Because many cancers and tumors continue to divide due in part to reduced or inactivated p53 function, compounds that restore p53-mediated cell apoptosis are useful as anti-cancer and anti-tumor therapies in humans. Such p53 activation compounds would function effectively as therapeutic agents for individuals suffering from cancer or tumor growth.

In addition, since overactivity of p53-mediated apoptosis may be the cause of autoimmune or neurodegenerative diseases, screening for modulators that reduce p53 activity also has utility in treating human disease. Therapeutically effective compositions of modulators that are found to reduce p53 activity could be given to patients as treatments for such diseases.

Definitions

“Modulation” refers to the capacity to either enhance or inhibit a functional property of biological activity or process (for example, p53 activity or binding); such enhancement or inhibition may be contingent on the occurrence of a specific event, such as activation of a signal transduction pathway, and/or may manifest only in particular cell types.

The term “modulator” refers to a chemical compound (naturally occurring or non-naturally occurring), such as a biological macromolecule (for example, nucleic acid, protein, non-peptide, or organic molecule), or an extract made from biological materials such as bacteria, plants, fungi, or animal (particularly mammalian) cells or tissues. Modulators are evaluated for potential activity as inhibitors or activators (directly or indirectly) of a biological process or processes (for example, agonist, partial antagonist, partial agonist, antagonist, antineoplastic agents, cytotoxic agents, inhibitors of neoplastic transformation or cell proliferation, cell proliferation-promoting agents, and the like) by inclusion in screening assays described herein. The activity of a modulator may be known, unknown or partially known.

The term “DNA damaging agent” is meant to encompass any agent that causes damage to DNA. Examples of such agents include chemicals, radioactive molecules and light, including ultraviolet light.

The term “transcriptional cofactor” is meant to encompass cofactors that work directly or indirectly with p53 to affect transcription from a target gene. Examples of such cofactors includes the histone acetyl transferase cofactor p300, Pit-1, MDM2, TAFs, TRAP the CREB binding protein (CBP) and p300 (CBP/p30O), and both CBP/p300 and p300/CBP-associated factor (PCAF).

The term “association” or “associated” is meant to encompass both direct and indirect interactions between several molecules.

The term “apoptotic promoter” is meant to encompass any promoter wherein activation or inhibition of the promoter leads to apoptosis of a cell. A “pro-apoptotic” promoter is a promoter that when activated leads to apoptosis in a cell.

The term “target gene” is meant to encompass genes that are bound by the p53 protein, or related complexes. Examples of target genes include p21, Mdm2, GADD45, Bax, or cyclin G. “Cell cycle target genes” are those target genes involved in regulating the cell cycle.

“Naturally-occurring” as used herein, as applied to an object, refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Operably linked” refers to a juxtaposition wherein the components so described are in a relationship permitting them to function in their intended manner. For instance, two genes which produce a fusion protein are operably linked.

“Polynucleotide” refers to a polymeric form of nucleotides of at least 10 bases in length, either ribonucleotides or deoxynucleotides or a modified form of either type of nucleotide. The term includes single- and double-stranded forms of DNA.

“Test compound” refers to a compound to be tested by one or more screening devices or method(s) of the invention as a putative modulator.

“Fluorescent label” refers to incorporation of a detectable marker, for example, by incorporation of a fluorescent moiety to a chemical entity that binds to a target or attachment to a polypeptide of biotinyl moieties that can be detected by avidin (for example, streptavidin containing a fluorescent label or enzymatic activity that can be detected by fluorescence detection methods). Various methods of labeling polypeptides and glycoproteins are known in the art and may be used. Examples of labels for polypeptides include, but are not limited to dyes (for example, FITC and rhodamine), intrinsically fluorescent proteins, and lanthanide phosphors. In some embodiments, labels are attached by spacer arms of various lengths to reduce potential steric hindrance.

“Reporter gene” refers to a nucleotide sequence encoding a protein that is readily detectable either by its presence or activity, including, but not limited to, luciferase, green fluorescent protein, chloramphenicol acetyl transferase, p-galactosidase, secreted placental alkaline phosphatase, β-lactamase, human growth hormone, and other secreted enzyme reporters. Generally, reporter genes encode a polypeptide not otherwise produced by the host cell which is detectable by analysis of the cell(s), for example, by the direct fluorometric, radioisotopic or spectrophotometric analysis of the cell(s) and preferably without the need to remove the cells for signal analysis of a well. Preferably, the gene encodes an enzyme which produces a change in fluorometric properties of the host cell which is detectable by qualitative, quantitative or semi-quantitative function of transcriptional activation. Exemplary enzymes include esterases, phosphatases, proteases (tissue plasminogen activator or urokinase) and other enzymes whose function can be detected by appropriate chromogenic or fluorogenic substrates known to those skilled in the art. Proteins, particularly enzymes, of reporter genes can also be used as probes in biochemical assays, for instance after proper conjugation to either the target or a chemical entity that binds to the target.

“Pharmaceutical agent” or “drug” refer to a chemical compound or composition capable of inducing a desired therapeutic effect when properly administered to a patient.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (for example, epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “disease state” refers to a physiological state of a cell or of a whole mammal in which an interruption, cessation, or disorder of cellular or body functions, systems, or organs has occurred.

The term “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “disorder” is any condition that would benefit from treatment of the present invention. This includes chronic and acute disorders or disease including those pathological conditions which predispose the mammal to the disorder in question. Non-limiting examples of disorders to be treated herein include benign and malignant tumors, leukemias and lymphoid malignancies, in particular breast, rectal, ovarian, stomach, endometrial, salivary gland, kidney, colon, thyroid, pancreatic, prostate or bladder cancer. A preferred disorder to be treated in accordance with the present invention is malignant tumor, for example, cervical carcinomas and cervical intraepithelial squamous and glandular neoplasia, renal cell carcinoma (RCC), esophageal tumors, and carcinoma-derived cell lines.

“Mammal” for purposes of treatment refers to any animal classified as a mammal, including humans, domestic and farm animals, and zoo, sports, or pet animals, such as dogs, horses, cats, cows, etc. Preferably, the mammal is human.

“Therapeutic formulations” of the p53 modulators are prepared for storage by mixing modulators having the desired degree of purity with optional physiologically acceptable carriers, excipients, or stabilizers (Remington: The Science and Practice of Pharmacy, 19th Edition, Alfonso, R., ed, Mack Publishing Co. Easton, Pa. 1995), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

A p53 modulator to be used for in vivo administration must be sterile. This is readily accomplished by filtration through sterile filtration membranes, prior to or following lyophilization and reconstitution. The p53 modulator ordinarily will be stored in lyophilized form or in solution.

Therapeutic p53 modulator compositions generally are placed into a container having a sterile access port, for example, an intravenous solution bag or vial having a stopper pierceable by a hypodermic injection needle.

The route of p53 modulator administration is in accord with known methods, for example, injection or infusion by intravenous, intraperitoneal, intracerebral, subcutaneous, intramuscular, intraocular, intraarterial, intracerebrospinal, or intralesional routes, or by sustained release systems as noted below. Preferably the modulator is given systemically.

Suitable examples of sustained-release preparations include semipermeable polymer matrices in the form of shaped articles, for example, films, or microcapsules. Sustained release matrices include polyesters, hydrogels, polylactides (U.S. Pat. No. 3,773,919: EP 58,481), copolymers of L-glutamic acid and gamma ethyl-L-glutamate (Sidman et al. 1983 Biopolymers 22:547-556), poly (2-hydroxyethyl-methacrylate) (Langer et al. 1981 J Biomed Mater Res 15:167-277; and Langer 1982 Chem Tech 12:98-105), ethylene vinyl acetate (Langer et al., supra) or poly-D-(−)-3-hydroxybutyric acid (EP 133,988). Sustained-release p53 modulator compositions may also include liposomally entrapped modulators. Liposomes containing modulators are prepared by well-known methods. Ordinarily the liposomes are of the small (about 200-800 Angstroms) unilamelar type in which the lipid content is greater than about 30 mol. % cholesterol, the selected proportion being adjusted for the optimal therapy.

A p53 modulator can also be administered by inhalation. Commercially available nebulizers for liquid formulations, including jet nebulizers and ultrasonic nebulizers are useful for administration. Liquid formulations can be directly nebulized and lyophilized powder can be nebulized after reconstitution. Alternatively, a p53 modulator can be aerosolized using a fluorocarbon formulation and a metered dose inhaler, or inhaled as a lyophilized and milled powder.

An “effective amount” of p53 modulator to be employed therapeutically will depend, for example, upon the therapeutic objectives, the route of administration, the type of p53 modulator employed, and the condition of the patient. Accordingly, it will be necessary for the therapist to titer the dosage and modify the route of administration as required to obtain the optimal therapeutic effect. Typically, the clinician will administer the p53 modulator until a dosage is reached that achieves the desired effect. The progress of this therapy is easily monitored by conventional assays.

The patients to be treated with the p53 modulator of the invention include preclinical patients or those with tumors, such as esophageal, pancreatic, colorectal tumors, carcinomas, such as renal cell carcinoma (RCC), cervical carcinomas and cervical intraepithelial squamous and glandular neoplasia, and cancers, such as colorectal cancer, breast cancer, lung cancer, and other malignancies. Patients are candidates for therapy in accord with this invention until such point as no healthy tissue remains to be protected from tumor progression. It is desirable to administer a p53 modulator as early as possible in the development of the tumor, and to continue treatment for as long as is necessary.

In the treatment and prevention of tumor-associated disorder by a p53 modulator, the composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the modulator, the particular type of modulator, the method of administration, the scheduling of administration, and other factors known to medical practitioners. The “therapeutically effective amount” of a modulator to be administered will be governed by such considerations, and is the minimum amount necessary to prevent, ameliorate, or treat the disorder, including treating chronic autoimmune conditions and immunosuppression maintenance in transplant recipients. Such amount is preferably below the amount that is toxic to the host or renders the host significantly more susceptible to infections.

As a general proposition, the initial pharmaceutically effective amount of the modulator administered parenterally will be in the range of about 0.1 to 50 mg/kg of patient body weight per day, with the typical initial range of modulator used being 0.3 to 20 mg/kg/day, more preferably 0.3 to 15 mg/kg/day. The desired dosage can be delivered by a single bolus administration, by multiple bolus administrations, or by continuous infusion administration of the modulator, depending on the pattern of pharmacokinetic decay that the practitioner wishes to achieve.

As noted above, however, these suggested amounts of the modulator are subject to a great deal of therapeutic discretion. The key factor in selecting an appropriate dose and scheduling is the result obtained, as indicated above. For example, the modulator may be optionally formulated with one or more agents currently used to prevent or treat tumors such as standard- or high-dose chemotherapy and hematopoietic stem-cell transplantation. The effective amount of such other agents depends on the amount of p53 modulator present in the formulation, the type of disorder or treatment, and other factors discussed above. These are generally used in the same dosages and with administration routes as used hereinbefore or about from 1 to 99% of the heretofore employed dosages.

Other chemistry terms herein are used according to conventional usage in the art, as exemplified by The McGraw-Hill Dictionary of Chemical Terms (ed. Parker, S., 1985, McGraw-Hill, San Francisco, incorporated herein by reference).

p53 Assay

As discussed above, one aspect of the invention is a method of determining binding of p53 protein to chromatin-bound target DNA. Because DNA in vivo is typically always found as chromatin, an assay that analyzes p53 protein binding to chromatin-bound DNA is thought to more accurately reflect actual binding properties in vivo.

Chromatin is the portion of the cell nucleus that contains all the DNA in the nucleus of animal or plant cells. A small amount of special DNA is also found in the mitochondria of the cell cytoplasm outside of the cell nucleus. DNA is virtually never found as a naked molecule in animal or plant cell nuclei. DNA is always found in association with histone proteins (soluble in acid solutions), HMG proteins (soluble in neutral saline), residual proteins (soluble in concentrated urea solutions), phosphoproteins (soluble in basic solutions), RNA species (soluble in aqueous phenol solutions), and lipid species (soluble in chloroform-methanol solutions). However, as used herein, the term “chromatin” does not require a complex of DNA and every compound in the nucleus. All that is required is DNA in at least an association with histone proteins.

Accordingly, one embodiment of the invention comprises testing compounds for their effect on chromatin-assembled target DNA using DNA extracts and purified core histones, with or without chromatin-assembly proteins. In one embodiment, chromatin assembly of target DNA is performed using Drosophila embryo cell-free extracts and purified core histones as previously described (Bulger, M. & Kadonaga, J. 1994 Methods Mol Genet 5:242-262). In addition, it is possible to check the quality of the reconstituted chromatin by micrococcal nuclease digestion to look for the presence of physiologically spaced nucleosomes.

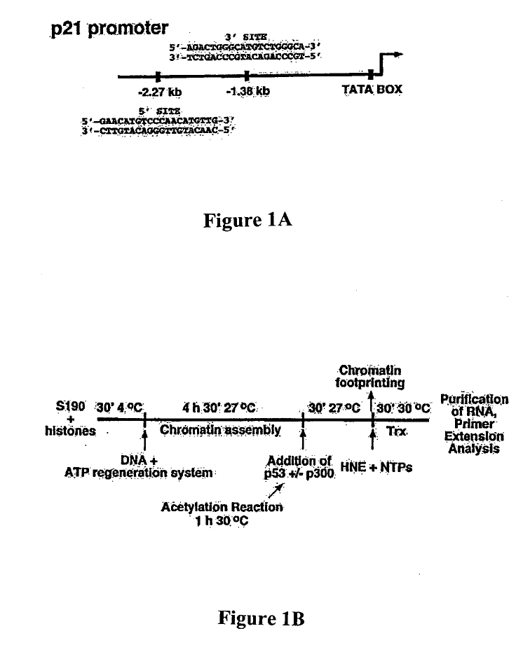

In one embodiment, a DNA construct containing a fragment of the human p21 promoter can be used as the p53 target DNA (FIG. 1A), since the p21 promoter is known to be activated by p53. In another embodiment, the promoter can be linked to a reporter gene, such as the luciferase reporter gene to create a reporter construct. Of course, one of skill in the art will recognize that other constructs and other reporter genes could be used and arc expected to function similarly.

Once a reporter construct is created, it can be subjected to in vitro transcription reactions using HeLa cell nuclear extracts as a source of basal initiation components in the presence or absence of p53 and/or additional factors such as p300, as described in the protocol depicted in FIG. 1B. The factors and components are preferably added after chromatin assembly is complete (“post assembly”) and incubated with the template prior to in vitro transcription. Gene expression is known in the art as the process by which a gene's coded information is converted into the structures present and operating in the cell. Expressed genes include those that are transcribed into mRNA and then translated into protein and those that are transcribed into RNA but not translated into protein.

Thus, using the aforementioned transcription assay, compounds can be determined that alter the activity of p53 in the presence of the test compound. Of course, embodiments of the invention are not limited to measuring p53 activity through in vitro transcription. Any method of protein chemistry that is known to a skilled artisan to measure activity of p53 is contemplated.

In yet another embodiment, an assay to measure the binding location of p53 protein to chromatin assembled target genes is performed by chromatin footprinting after addition of p53 protein and a compound of interest. In addition, basal initiation components, such as from HeLa cell nuclear extracts can be used in the assay. In one embodiment, the footprinting assay is a DNase I chromatin footprinting assay, as described in detail below in Example 1. Briefly, the chromatin assembled target gene is treated with DNase I in order to digest the DNA that is not protected by bound protein. The digested target DNA is then analyzed to determine which DNA sequences were protected from digestion. Accordingly, the binding sites of the p53 protein, and any associated factors can be determined.

In another embodiment, Electrophoretic Mobility Shift Assay (EMSA) testing of complex formation of p53 protein and target DNA is performed in the presence of a compound of interest. In yet another embodiment, the p53 protein, or the gene of interest is made to contain a radioactive marker, and the radioligand binding method, well known to a skilled artisan, is performed to measure the binding of p53 protein to the gene of interest in the presence of a compound of interest.

Thus, one embodiment is a test system that comprises a p53 protein and a chromatin-assembled DNA, or DNA of interest. While the use of human DNA is most preferred, the invention is not limited to the use of the human DNA. The corresponding DNA from other species can also be used. However, it is preferred that both the p53 protein and DNA used in the assay originate from the same species.

In another embodiment, the test system additionally comprises a promoter, preferably a pro-apoptotic promoter. In another embodiment, the test system additionally comprises a tissue extract comprising various transcription factors. The tissue may be from a disease-free animal, including human, or an animal affected with a neurodegenerative, neoplastic, or autoimmune disorder.

p53 Activity is Complex-Specific

Moreover, embodiments of the invention relate to the discovery that p53 recruits different activation complexes depending on the stresses put on the particular target cell. For example, p53 may recruit one activation complex in order to cause apoptosis in response to carcinogenic chemicals, but a completely different activation complex to cause apoptosis in response to radioactivity. For this reason, it is not only advantageous to screen for molecules that increase binding and activity of p53, but it is also advantageous to determine the identity of the molecules involved in each of the p53 activation complexes, so that compounds having an affect on those molecules can be determined. In addition, it is likely that molecules which affect a particular complex will not have an effect on another complex. Accordingly, in order to determine the most effective molecules for treating a particular p53-mediated indication, one can build a list of compounds that are effective for restoring p53 function in different cancers. Thus, when a patient is found to have a particular cancer, the appropriate molecule can be delivered to the patient which is known to increase p53 function, and thereby apoptosis, of the cancer cells.

As described in Example 2, a battery of genes that are transcriptionally activated in response to DNA damage by the tumor suppressor protein p53 in human osteosarcoma (U2OS) cells were analyzed to understand the mechanism by which apoptosis is regulated. For example, the p21 gene was found to be activated by p53 immediately after DNA damage or other forms of cellular stress to initiate cell cycle arrest. At later times, other specific pro-apoptotic genes were activated by p53 to induce cell death if the damage is too severe for the cell to repair.

Interestingly, different subsets of pro-apoptotic genes were activated by p53 in distinct tissues and in response to different forms of stress. Thus, cell death is normally controlled in a tissue- and stress-specific manner. Accordingly, embodiments of the invention include identifying compounds that are able to modulate the tissue- and stress-specificity of cell death in human cancers in order to spare normal cells from the harsh and non-discriminate effects of current chemotherapeutic treatments which kill all dividing cells. In addition, embodiments of the invention provide a means for identifying compounds that are able to modulate the inappropriate cell death that occurs in autoimmune and neurodegenerative diseases. Tumor cells fail to normally apoptose or eliminate themselves whereas in autoimmune and neurodegenerative diseases, cells spontaneously apoptose, thus the ability to both promote and inhibit apoptosis in certain tissues would be greatly advantageous.

To address this issue, a chromatin immunoprecipitation (ChIPs) technique was used to examine the temporal order of interaction of a variety of transcription proteins to individual p53-responsive promoters that control cell cycle arrest and apoptosis before and after DNA damage by two different damaging agents. The regulation of genes that control apoptosis after both UV irradiation and treatment with doxorubicin (which inhibits topoisomerase) was found to be at the level of recruitment of specific transcription initiation-proteins. For example, before damage the p21 promoter is already poised for transcription by containing bound RNA polymerase II, TATA binding protein (TBP), TFIIB, and TFIIH. This indicated that activation of cell cycle arrest by p21 gene expression was at the level of transcriptional elongation or “release” of a poised RNA polymerase II.

By contrast, pro-apoptotic promoters such as PTEN, FAS/APO1, PUMA, and p53AIP1 contained very low levels of bound initiation factors indicating that induction of apoptosis by pro-apoptotic gene expression is more likely to occur at the level of transcriptional initiation by recruitment of such factors as RNA polymerase II, TBP, TFIIB, and TFIIH.

After DNA damage by UV irradiation, a significant increase in the binding of phosphorylated p53 to the p21 promoter and an active recruitment of a specific component of the general transcription machinery, TAFII250 was observed. Both events were correlated with the induction of p21 gene expression. Surprisingly, after DNA damage by doxorubicin the binding of phosphorylated p53 to the p21 promoter was significantly increased. However, an active recruitment of another specific component of the general transcription machinery, TFIIB, and no recruitment of TAFII250 was observed.

This demonstrated that the differential recruitment of specific transcriptional initiation factors TFIIB and TAFII250 to the p21 promoter was stress-specific. In addition, this same phenomenon of stress-specific recruitment of TAFII250 after UV irradiation but not in response to doxorubicin was observed for the pro-apoptotic promoter FAS/APO1 even though the FAS/APO1 gene was expressed to approximately the same extent in response to each type of DNA damage.

By identifying stress- and tissue-specific differences in protein recruitment to the subset of genes that control apoptosis, compounds can be screened for those that: 1) selectively inhibit formation of the initiation complex on pro-apoptotic promoters in autoimmune and neurodegenerative diseases to prevent inappropriate cell death; and 2) selectively enhance formation of the initiation complex on pro-apoptotic promoters in tumors to promote cell death.

Accordingly, embodiments of the invention include in vitro assays using plasmids containing the recombinant human p21 promoter or other human pro-apoptotic promoters, such as FAS/APO1, to examine differential recruitment of transcriptional initiation complexes from protein extracts derived from tumor cells of different tissue origin after treatment with specific types of DNA damaging agents.

Another embodiment of the invention is the use of protein extracts from cells affected by autoimmune disorders or neurodegenerative diseases in order to determine the recruitment of specific p53 complexes in these diseases. Protein recruitment is examined on plasmids as both DNA and when assembled into chromatin. In addition, the requirement of promoter-bound phosphorylated p53 for selective recruitment of factors such as TAFII250 can also be employed in this assay. Small molecule compounds can be screened using variations of this assay to preferentially inhibit or enhance formation of stress- and tissue-specific transcription initiation complexes on individual promoters or subsets of promoters that control apoptosis in distinct tissues.

As used herein, “p53” and “p53 protein” can be a recombinant form of p53, a synthesized form of p53, or purified from an original organism. p53 can be directly purified from various sources (for example from bacteria, baculovirus or mammalian cells). It is, however, not necessary to use a full-length p53 for performing the assay of the present invention. Accordingly, the p53 form used herein also means any useful variant or fragment of p53, preferably of human p53. The features of such a useful variant or fragment are clear from the description hereinafter.

When using the assay, p53 preferably forms complexes with chromatin-associated DNA. Consequently, for DNA binding an active p53 DNA binding domain (for example, residues 102-292 of p53) should be present in the p53 form used for the assay. p53 protein can be in its native unmodified form. Alternatively, p53 protein may be modified by acetylation, or by binding to a specific antibody (for example, the monoclonal antibody PAb421 known in the art to bind to the amino acid stretch between amino acid 372 and 380 of the human p53), or by phosphorylation by kinases (casein kinase II phosphorylating Ser392 of human p53 or protein kinase C phosphorylating Ser 370 and Ser 375 of human p53), or by truncation of its C-terminus (deletion of maximal 38 amino acids of the C-terminus of the natural p53 sequence). An example for the latter is p53A30, that is, natural p53 lacking the C-terminal 30 amino acids, used hereinafter. Alternatively, p53 protein may be mutated. The mutated p53 may be recombinant, or purified from a sample obtained from a malignant tissue.

The amount of p53 that is preferentially used in the assay depends on the amount of chromatin-assembled DNA used in the assay. Typically, for 500 ng of DNA template in chromatin the amount of p53 added is preferably between about 0.1 pmol and about 50 nmol. However, it is also possible to use other ratios of DNA to p53 to obtain a clear signal in the subsequent product detection assay, for example, in vitro transcription assay.

The DNA element of the test system can be any DNA fragment which specifically binds to p53, for example, such as containing a p53 binding element degenerated or not. It can be a synthetic oligonucleotide, a DNA fragment isolated from a living organism or a DNA element inserted in a plasmid. The length of the DNA fragment may be between 10 bp to 2,000 kb. Some genes that are identified as being regulated by p53 are described, for example, in Zhao, R. et al. 2000 Genes and Dev 14:981-993; and US application 2001/0039013, incorporated herein by reference in their entirety.

The in vitro transcription, Electrophoretic Mobility Shift Assay (EMSA), and chromatin and DNA footprinting assays are performed using methods well known to a skilled artisan.

The compounds which may be screened in accordance with the invention include, but are not limited to peptides, antibodies and fragments thereof, and other organic compounds (for example, peptidomimetics) that associate with p53 bound to specific chromatin-associated DNA binding sites and either mimic the activity triggered by the natural transcriptional co-activator or co-repressor or inhibit the activity triggered by the natural transcriptional co-activator or co-repressor; as well as peptides, antibodies or fragments thereof, and other organic compounds that modulate p53 interaction with specific co-factors that selectively associate with p53 bound to chromatin-assembled DNA.

Such compounds may include, but are not limited to, peptides such as, for example, soluble peptides, including but not limited to members of random peptide libraries; (see, for example, Lam, K. S. et al. 1991 Nature 354:82-84; Houghten, R. et al. 1991 Nature 354:84-86), and combinatorial chemistry-derived molecular library made of D- and/or L-configuration amino acids, phosphopeptides (including, but not limited to, members of random or partially degenerate, directed phosphopeptide libraries; see, for example, Songyang, Z. et al. 1993 Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened include, but are not limited to, small organic molecules that are able to cross the blood-brain barrier, gain entry into an appropriate cell (for example, in the cancerous cells or cell of an individual with detected p53 mutation) and enhance the binding of mutated p53 to its various target genes.

Computer modeling and searching technologies permit identification of compounds, or the improvement of already identified compounds, that can modulate p53 binding or activity. Having identified such a compound or composition, the active sites or regions are identified. Such active sites might typically be ligand-binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential p53 modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al. 1988 Acta Pharmaceutical Fennica 97:159-166; Ripka 1988 New Scientist 54-57; McKinaly and Rossmann 1989 Annu Rev Pharmacol Toxicol 29:111-122; Perry and Davies 1989 OSAR: Quantitative Structure-Activity Relationships in Drug Design pp. 189-193 Alan R. Liss, Inc; Lewis and Dean 1989 Proc R Soc Lond 236:125-140 and 141-162; and, with respect to a model receptor for nucleic acid components, Askew, et al. 1989 J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario).

Although described above with reference to design and generation of compounds which could alter binding, one could also screen libraries of known compounds, including natural products or synthetic chemicals, and biologically active materials, including proteins, for compounds which are inhibitors or activators.

Another embodiment is a test kit for testing the effect of a substance on the binding of p53 to chromatin-assembled DNA. The test kit preferably includes: (a) a p53 or functional equivalent or fragment thereof having a DNA-binding domain, (b) a chromatin-associated DNA sequence that specifically binds to the p53 binding domain. In another embodiment the test kit includes (a) a p53 or functional equivalent or fragment thereof having DNA-binding domain, (b) a DNA sequence specifically binding to the p53 binding domain, and (c) purified core histones. The test kit optionally contains instructions for its use.

Embodiments of the invention also relate to the discovery that, using the natural p21 promoter, p53 functions were found to act synergistically with p300 to activate transcription through chromatin from a distance of at least 1.4 kb. Interestingly, p300 mediates p53-dependent transcription without increasing p53 binding to the chromatin template. Instead, p53 associates with its nucleosomal target sites in the absence of chromatin remodeling or modifying complexes. Chromatin-bound p53 then recruits p300 to the p21 promoter, resulting in localized nucleosome acetylation with regional spreading to the TATA box. p300 mediates p21 gene expression by p53-targeted nucleosomal acetylation rather than through p53 acetylation, which does not affect its transcriptional activity in vitro.

In contrast with current views, p53 does not appear to be a latent, but is rather, an active DNA binding protein that does not require modification of the C-terminal domain by acetylation or antibody binding to interact with either DNA or chromatin.

Studies indicate that the C terminus is not inhibitory for p53 binding and in fact, appears to be required for p53 association with particular target sites. p53 interacts with distinct types of nucleosomal binding sites within the p21 promoter, as shown by different behaviors in response to C terminus perturbations. In addition, p53 associates with chromatin at higher affinity than with DNA in the absence of cofactors or protein modifications. Thus, transcriptional regulation by p53 may be a complex property of chromatin structure, DNA topology, and recruitment of specific cofactors to allosterically regulated binding sites.

### Synergistic Transcriptional Activation of the Chromatin-Assembled p21 Promoter by p53 and p300

Full-length human p53 and full-length human p300 were transfected into in insect cells using the baculovirus expression system. Flag-tagged p53 and His-tagged p300 were purified by affinity chromatography to apparent homogeneity. The full-length size of recombinant p300 was confirmed by comparative Western blot, using native p300 as control. To assay p300 acetyltransferase activity, p300 was incubated with p53 or free core histones in the presence of ³H-acetyl CoA. The products of these reactions were then electrophoresed and visualized by Coomassie staining or fluorography to detect acetylated proteins. p300 efficiently acetylated p53 and histones H3 and H4 in solution and histones H2A and H2B to a lesser extent. These results show that full-length p300 can acetylate p53, as previously reported for its histone acetyl-transferase (HAT) domain alone (Gu, W. and Roeder, R. G. 1997 Cell 90:595-606).

Equal amounts of native or acetylated p53 were then incubated with a ³²P labeled 25 bp oligonucleotide containing a consensus p53 binding site (p21 promoter, 5′ site) and analyzed by Electrophoretic Mobility Shift Assay (EMSA). The p53 protein obtained from insect cells was mainly inactive for specific DNA binding activity when measured by EMSA using a 25 bp oligonucleotide harboring the 5′ site in the p21 promoter. In agreement with previous experiments using only the catalytic domain of p300, the DNA binding activity of p53 was dramatically improved by acetylation using full-length p300. The efficiency of activation by p300-mediated acetylation was comparable to that observed with other known activating stimuli, such as binding of the antibody PAb421 to the C-terminal region of the protein or deletion of the C terminus itself. These experiments demonstrate that the p53 protein produced in insect cells is mainly nonacetylated, an observation that is further supported by Western blot assays using antibodies that specifically recognize the acetylated form of p53. This experiment also indicates that other modifications previously reported to activate p53 DNA binding activity are absent in these preparations.

To analyze the mechanism of p53-dependent transcriptional activation of chromatin-assembled p21 promoters, a DNA construct containing a 2.4 kb fragment of the human p21 promoter was used. This included two binding sites for p53, which are centered around positions −2270 (5′ site) and −1380 (3′ site), respectively (FIG. 1A). This promoter fragment drives the transcription of a luciferase reporter gene (WWP-LUC or p21-LUC). This 8 kb plasmid has been extensively used to study p53-dependent transcription of the p21 promoter by transient expression experiments, and the requirement of at least one of these two binding sites for p53-dependent transactivation has been amply demonstrated (El-Deiry, W. S. et al. 1995 Cancer Res 55:2910-2919).

Chromatin assembly of this template DNA was performed using Drosophila embryo cell-free extracts and purified core histones as previously described (Bulger, M. and Kadonaga, J. 1994 Methods Mol Genet 5:242-262). The quality of the reconstituted chromatin was assessed by transcriptional repression and structural analysis by micrococcal nuclease digestion. The micrococcal nuclease digestion pattern of the in vitro reconstituted chromatin showed the presence of physiologically spaced nucleosomes and was correlated with a complete repression of p21 promoter activity.

The p21-LUC chromatin template was subjected to in vitro transcription reactions using HeLa cell nuclear extracts as a source of basal initiation components in the presence or absence of p53 and/or p300, as described in the protocol depicted in FIG. 1B. Both factors were added after chromatin assembly was complete (“post assembly”) and incubated with the template prior to in vitro transcription. Addition of p53 alone produced very modest transcriptional activation. However, coincubation with p300 gave a strong synergistic de-repression of the chromatin template. p300 alone was unable to achieve such activation. Neither p53 nor p300 appreciably influenced transcription of naked DNA templates. Identical results were obtained using extracts from another cell line, erythroid K562. Thus, transcriptional activation from the chromatin-assembled p21 promoter in vitro requires p53, acting at a distance of at least 1.38 kb, in combination with a histone acetyl transferase cofactor, p300. Interestingly, other putative cofactors, such as PCAF and human SWI/SNF, do not facilitate p53-dependent transcription in these assays even though they both interact with p53.

### p300 Can Mediate p53-Dependent Transcription without Increasing p53 Binding to the Chromatin Template

To analyze the mechanism by which p300 mediates p53-dependent activation, DNase I chromatin footprinting assays were performed to examine the occupancy of the 5′ and 3′ sites of the p21 promoter under conditions of transcriptional coactivation. Chromatin assembly and factor incubation steps were performed as described above, but the samples were split into three aliquots just before the in vitro transcription reaction. One aliquot was subjected to DNase I digestion, the second was transcribed, and the third was analyzed by Western blot to determine the acetylation status of p53. Surprisingly, p53 alone was able to generate a clear footprinting pattern at both the 5′ and 3′ binding sites within the p21 promoter. Addition of p300 did not significantly change the footprinting pattern produced by p53, even though it clearly enhanced transcription from the p21 promoter. In a parallel experiment using the same proteins and reagents, p300 clearly activated p53 DNA binding activity in EMSA.

The acetylation status of both untreated and previously acetylated p53 was preserved during incubation with the chromatin template as determined by Western blot analyses using antibodies that specifically recognize only the acetylated form of p53. These antibodies did not recognize native p53, whereas the p300-acetylated p53 isoform gave a strong signal. Thus, native p53 remained unacetylated, and previously acetylated p53 was not deacetylated during the chromatin assembly incubation. Accordingly, this experiment demonstrates that p300 can mediate p53 transactivation without affecting the ability of p53 to bind to its DNA recognition sequences in chromatin.

### p53 Does not Require Acetylation or Remodeling Complexes to Interact with Chromatin

DNA footprinting experiments were performed with chromatin-assembled p21 promoters after ATP depletion and purification by gel filtration using nonsaturating amounts of p53. These chromatin samples are devoid of endogenous ATP-dependent nucleosome remodeling activities, small molecules like ATP or acetyl CoA, and the majority of proteins from the chromatin assembly extract (Mizuguchi, G. and Wu, C. 1999 in: Peter Becker (Ed.) Chromatin Protocols. Totowa, N.J.: Humana Press). Both native and previously acetylated p53 were found to be able to interact with the 5′ and 3′ binding sites in the purified p21 promoter chromatin with similar affinities.

From these results, three main conclusions could be drawn. First, although native p53 possesses an almost undetectable DNA binding activity when assayed on short oligonucleotides, it binds very efficiently to the same sites in a larger promoter context when assembled into chromatin. Second, whereas acetylation of p53 by p300 dramatically increases its affinity for short DNA duplexes, it does not increase its affinity for the same sequences in p21 promoter chromatin. Third, native p53 interacts efficiently with its nucleosomal binding sites in the absence of chromatin modifying or remodeling activities.

### p53 Possesses Distinct Types of Chromatin and DNA Binding Sites

To gain more insight into the interaction of p53 with chromatin, a different tool commonly employed to activate its dormant DNA binding activity was used. Previous reports have concluded that the C-terminal domain of p53 inhibits its ability to interact with DNA. Such inhibition can be artificially relieved by binding a monoclonal antibody to the C-terminal domain (Hupp, T. R. et al. 1992 Cell 71:875-886). Interestingly, the binding of the monoclonal antibody PAb241 did not increase the affinity of p53 for all DNA binding sites tested. Moreover, the affinity for some binding sites is significantly decreased, whereas it remains unchanged for others. Similar behavior has been observed for other “activating” modifications, suggesting the existence of different types of DNA binding sites for p53. In this regard, the two p53 recognition sequences in the p21 promoter fall into different categories: binding to the 5′ site is strongly enhanced by treatment with PAb421, whereas binding to the 3′ site is inhibited when measured by EMSA (Resnick-Silverman, L. et al. 1998 Genes & Dev 12:2102-2107). Therefore, the effect of PAb421 on p53 binding to these two binding sites was tested when assembled into chromatin.

The addition of the antibody PAb421 to native p53 increases its DNA binding activity for the 5′ site to a similar extent as that observed by p300-dependent acetylation when examined by EMSA. As expected, the protein-DNA complex was supershifted by the presence of the antibody. However, when examined by chromatin footprinting, treatment with PAb421 did not increase the affinity of p53 for the 5′ site, similar to our observations with acetylation by p300. Interestingly, PAb421 clearly blocked the binding of p53 to the 3′ site. Thus, using chromatin-assembled p21 promoters it was found that the antibody retained its inhibitory effect on p53 interaction with the 3′ site, but it lacked the stimulatory effect on the 5′ site, in agreement with previous reports (Kim, E. et al. 1997 Oncogene 15:857-869; Cain, C. et al. 2000 J Biol Chem 275:39944-39953).

This chromatin footprinting analysis allows indicated two major conclusions. First, two different putative activating modifications of native p53, namely acetylation and binding of the antibody PAb421, fail to improve the ability of p53 to interact with the chromatin-assembled p21 promoter. Second, different types of p53 binding sites exist within this promoter as revealed by the selective inhibition of binding to only the 3′ site by PAb421.

### The C-Terminal Domain of p53 is not Inhibitory and is Differentially Required for Binding to Distinct DNA Sequences

The observation that two modifications of the C-terminal domain, acetylation and binding of the antibody PAb421, do not enhance the interaction of p53 with the p21 promoter argues against the idea that the C-terminal domain inhibits the DNA binding activity of p53. To gain insight into this issue, a C-terminal deleted form of p53, lacking the last 30 amino acid residues (p53ΔC30) was investigated. After production in insect cells and comparative quantification with wild-type p53, the binding activity of this protein was tested in both EMSA and DNase I footprinting. As expected according to previous reports, the ΔC30 mutant was strongly active in the EMSA analysis as compared with the acetylated form of wild-type p53. By contrast, when the ΔC30 mutant was assayed by DNase I footprinting on p21 promoter DNA or chromatin, it showed a weaker binding to the 5′ site and no binding to the 3′ site. Thus, it appears that the C-terminal domain is not inhibitory for DNA binding and that it is required for interaction with both the 5′ and 3′ p21 promoter sites (to different extents) when these are present in larger molecules of DNA or chromatin. This is consistent with a functional analysis showing that the C-terminal domain is required for in vitro transcription from the chromatin-assembled p21 promoter.

The failure to detect latent DNA binding activity of p53 that can be activated by acetylation, incubation with the PAb421 antibody, or deletion of the C-terminal domain raises an important issue. Such latency is clearly evident when measuring p53 DNA binding activity by EMSA using short duplexes of naked DNA, but it is not apparent when the same binding sites exist within a larger DNA context with or without chromatin assembly. The resolution of this discrepancy is critical toward understanding the regulation of p53 DNA binding activity.

### The Affinity of p53 for the 5′ p21 Promoter Binding Site Increases as a Function of Target DNA Size

There are many differences between the two DNA binding assays performed in this study, EMSA and chromatin or DNA plasmid footprinting. First, the size of the template DNA is much longer in the footprinting experiments (25 bp for EMSA versus 8 kb for footprinting). Second, protein-DNA binding occurs in solution in footprinting reactions, whereas binding continues inside the polyacrylamide gel in EMSA, which can affect the formation or stability of the complex.

To discriminate between these alternatives, EMSA was performed with native and acetylated p53 using pieces of double-stranded naked DNA of increasing size. Probes were generated that contained a centered 5′ site of the p21 promoter with increasing flanking regions from the same promoter which are devoid of any other p53 binding sites. As a result, binding of p53 to a 25 bp oligonucleotide was increased 12-fold by acetylation, whereas binding to a 160 bp DNA molecule was increased only 1.5-fold. This decrease in the influence of acetylation was due to the fact that unacetylated p53 had increased affinity for the longer piece of DNA. The presence of additional p53 binding sites in the longer molecule is unlikely because only one shifted complex in EMSA was detected, and only one protected site within this region in a parallel footprinting experiment was found. The lack of effect of acetylation on p53 binding is most apparent when analyzed on the p21 promoter within an 8 kb plasmid. In this case, native and acetylated isoforms of p53 showed indistinguishable affinities for the 5′ binding site as determined by DNase I footprinting.

These experiments indicated that p53 has very low affinity for its recognition sequences when present in the form of short oligonucleotides, and that the binding improves significantly when the flanking regions are extended. This type of behavior is very suggestive of the presence of secondary structures in the binding sites (Kim, E. et al. 1997 Oncogene 15:857-869). Such structures cannot be adopted by short oligonucleotides, but are firmly stabilized by the addition of adjacent duplex regions. An alternative explanation is the possible requirement of DNA bending at the p53 binding sites for high affinity protein interaction (Nagaich, A. K. et al. 1999 PNAS USA 96:1875-1880). Interestingly, as the affinity of p53 increases for DNA of a particular length and topology, it becomes indistinguishable from the acetylated form.

### p53 Binds with Higher Affinity to Chromatin than to DNA

The relative binding affinities of p53 was then addressed for its two sites within the p21 promoter as DNA or chromatin. The interaction of some DNA binding proteins was impeded by nucleosomes, and specific remodeling complexes were required to facilitate their association (Armstrong, J. A. et al. 1998 Cell 95:93-104; Kadam, S. et al. 2000 Genes & Dev 14:2441-2451). To address this issue for p53, extensive protein titrations were performed in parallel experiments using DNA or CL4B-purified chromatin. Surprisingly, these experiments revealed that p53 binds with higher affinity to nucleosomal templates, most significantly at the 5′ site. Binding conditions were exactly the same for both DNA and chromatin templates, and protein concentrations were equalized with purified BSA to avoid “protein carrier” effects. DNA concentration was also the same in all reactions as assessed by electrophoresis in agarose gels. Because DNA supercoiling strongly influences p53 binding within the MDM2 promoter (Kim, E. et al. 1999 Oncogene 18:7310-7318), the relaxation introduced by nucleosome assembly was examined to explain the difference in the affinity for naked DNA compared to chromatin. However, after relaxing supercoiled p21-LUC plasmids by treatment with topoisomerase I, no differences in p53 binding to the 5′ or 3′ site were observed between relaxed and supercoiled templates.

Quantification of the occupancy of both 5′ and 3′ p21 promoter sites was performed by densitometric analysis of bands. This demonstrated a clear difference in the kinetics of p53 binding to DNA or chromatin templates at both sites. Whereas p53 interaction with chromatin increased almost logarithmically linear with protein concentration, binding to DNA showed a clear “threshold” effect with a dramatic change in occupancy within a narrow window of protein concentration. This difference was more apparent for the 3′ site. Taken together, these data indicate that the structure of the two p21 promoter binding sites may be altered within nucleosome arrays to generate a higher affinity for p53 and to influence its association kinetics. This regulatory feature of chromatin could be of critical importance within cells when distinct p53 target genes are activated in response to changing levels of p53 protein.

### Acetylation of the C-Terminal Domain of p53 Does not Influence Its Transcriptional Activity

Gene regulation by p53 occurs in discrete stages: first, interaction with its binding sites in chromatinized target promoters; and second, transactivation of the transcriptional machinery. After observing that p300 is required to mediate p53-dependent activation of chromatin-assembled p21 promoters, but does not facilitate the binding of p53, the acetylation of p53 was investigated to determine whether it played an important role at another step in the transcription process. The acetylation pattern of p53 has been previously determined, and mutations of lysine residues that are targeted by p300 have been generated and analyzed elsewhere (Gu, W. and Roeder, R. G. 1997 Cell 90:595-606; Avantaggiati, M. L. et al. 1997 Cell 89:1175-1184; Scolnick, D. M. et al. 1997 Cancer Res 57:3693-3696; Luo, J. et al. 2000 Nature 408:377-381). A mutant version of p53 was then analyzed in which lysines 370, 372, 373, 381, and 382 within the C-terminal domain were replaced by arginine (p53KR, Luo, J. et al. 2000 Nature 408:377-381). p53KR was expressed in insect cells, purified, and analyzed for its ability to activate transcription compared with wild-type p53. The mutant protein was still coactivated by p300 to a similar extent as wild-type p53. This indicates that acetylation of the C-terminal domain does not play a major role in either chromatin binding or subsequent steps in the p300-mediated activation process. A Western blot analysis confirmed that p53KR is not recognized by antibodies specific to acetylated p53. These results are in agreement with in vivo transient expression experiments, which demonstrate that mutations in acetylatable lysines result in only a slight or no decrease in p53-dependent transactivation (Scolnick, D. M. et al. 1997 Cancer Res 57:3693-3696; Nakamura, S. et al. 2000 Mol Cell Biol 20:9391-9398).

### p53 Recruits p300 and Directs Nucleosome Acetylation of the p21 Promoter

These studies indicate that p300 does not coactivate p53 function by either facilitating its interaction with chromatin or increasing its transactivation potential. Thus, other mechanisms were explored by which p300 may mediate p53-dependent transcription. Particularly, whether p53 recruits p300 to acetylate nucleosomal histones within the p21 promoter was analyzed. For this purpose, the p21 promoter was assembled into chromatin using defined factors instead of Drosophila embryo extracts. This recombinant chromatin assembly system, developed by Kadonaga and colleagues, consists of the ACF complex (dACF+dISWI) and the histone chaperone NAP-1, which together catalyze the assembly of free histones into nucleosomes in the presence of ATP. This system has been used successfully to obtain regularly spaced nucleosome arrays and allows a higher degree of manipulation compared to the embryo extracts (Ito, T. et al. 1999 Genes & Dev 13:1529-1539).

Use of recombinant factors enabled detection of de novo acetylation of the chromatin-assembled p21 promoter by incorporation of exogenously added ³H-acetyl CoA. Under the optimal histone:DNA ratio, the majority of histones in the assembly reaction were incorporated into a nucleosomal array. This is interesting because it has been demonstrated recently that p300 alone can acetylate free histones but not nucleosomes unless recruited by the activator GAL4-VP16 (Ito, T. et al. 2000 Genes & Dev 14:1899-1907).

The quality of p21 promoter chromatin assembled in this purified system was determined by micrococcal nuclease digestion, as for embryo extracts, with similar results. The templates were then incubated with p53 and/or p300 in the presence of ³H-acetyl CoA and the extent of acetylation monitored by incorporation of radioactivity when analyzed by SDS-PAGE and fluorography. When both p53 and p300 were present in the reaction, a strong acetylation of all four nucleosomal histones was easily detected, whereas very weak acetylation was observed by p300 alone. A control reaction with equivalent amounts of free histones and p300 was included. Acetylation of p53 and autoacetylation of p300 was also detected. This experiment clearly demonstrates that p53 can recruit the HAT activity of its coactivator p300 to the chromatin template and mediate de novo acetylation of nucleosomal histones.

To determine whether p53 influenced the localization of nucleosome acetylation on the p21 promoter by direct recruitment of p300, in vitro chromatin immunoprecipitation assays were performed. p21 promoters were assembled into chromatin using purified components and incubated with p53 and/or p300. Chromatin was digested with micrococcal nuclease and acetylated oligonucleosomes were immunoprecipitated using anti-acetyl-H4 antibodies. Specific p21 plasmid sequences in the immunoprecipitates were detected by PCR amplification or Southern blot. Identical results were obtained from both assays. As expected, the amount of DNA precipitated was greatly enhanced when both p53 and p300 were incubated with the chromatin template. The extent of increased acetylation in different regions of the p21-LUC plasmid varied from 3.4-fold near the 3′ p53 binding site to 1.4-fold in a distal region located 2 kb downstream of the proximal promoter. Interestingly, nucleosome acetylation also occurred several hundred base pairs from the 3′ site to reach the proximal promoter (TATA box). The following decreasing levels of acetylation were found: 3′ site>TATA box=5′ site>>+2 kb.

These data indicate that in the presence of p53, p300 acetylates nucleosomes in a targeted manner within chromatin-assembled p21 promoter-containing plasmids. In the absence of p53, only negligible nucleosome acetylation by p300 is apparent. Interestingly, acetylation was highest when p300 was recruited to the promoter proximal 3′ p53 binding site and then apparently spread to encompass the TATA box. Thus, the probable mechanism by which p300 coactivates p53-dependent transcription is by targeted nucleosomal acetylation of the proximal promoter when recruited by bound p53 at a distance of at least 1.4 kb.

It thus appears that p53 activates transcription from a natural target promoter, p21, when bound at a distance of at least 1.4 kb in a chromatin environment. Transcriptional activation appears to require the histone acetyltransferase, p300. Surprisingly, p300 does not function by facilitating p53 binding to its DNA recognition sites within chromatin. Instead, p300 acts at a later step in the transcription process by acetylating nucleosomes within the proximal and distal p21 promoter when targeted by bound p53. This presumably renders the nucleosomes sufficiently fluid to allow interaction with other components of the transcription machinery. p300-mediated transcriptional activation has been described for other chromatin-assembled genes (Kraus, W. L. et al. 1999 Mol Cell Biol 19:8123-8135; Dilworth, F. J. et al. 1999 PNAS. USA 96:1995-2000).

These experiments demonstrate that a mechanism by which p300 can regulate the activity of natural promoters is by acetylating chromatin over a long-range when recruited by a distal transcription factor. Targeted nucleosome acetylation by chromatin-bound GAL4-VP16 has been described (Ito, T. et al. 2000 Genes & Dev 14:1899-1907). Previous studies in yeast revealed a localized factor-directed acetylation encompassing 1-2 nucleosomes within the proximal promoters (Rundlett, S. E. et al. 1998 Nature 392:831-835; Struhl, K. et al. 1998 Cold Spring Harb Symp Quant Biol 63:413-421). In the absence of p53, p300 cannot acetylate nucleosomes due to lack of template targeting, and the p21 promoter remains inactive. It appears that p53 proteins containing mutations in lysine residues acetylated by p300 are as active as wild-type p53 in regulating p21 transcription in vitro. This indicated that acetylation of p53 does not contribute to its transactivation potential, and that p300 does not mediate transcription by this mechanism in our biochemical assays. This conclusion is in agreement with previous in vivo analyses in which p53 mutants lacking these lysine residues did not show a significant decrease in transcriptional activity (Scolnick, D. M. et al. 1997 Cancer Res 57:3693-3696; Nakamura, S. et al. 2000 Mol Cell Biol 20:9391-9398). However, p53 acetylation may play a role in protein stabilization or subnuclear localization (Nakamura, S. et al. 2000 Mol Cell Biol 20:9391-9398; Pearson, M. et al. 2000 Nature 406:207-210).

Numerous studies have focused on the DNA binding properties of p53 and the role of distinct protein domains in this process. This issue is especially germane because the majority of p53 mutations found in human cancers occur within the DNA binding domain. Thus, the inability of p53 to interact with its target genes and regulate transcription would be expected to contribute significantly to cancer development and progression. Previous experiments have led to the conclusion that p53 is a latent DNA binding protein which contains an inhibitory C-terminal domain. Using primarily EMSA analyses, latent DNA binding of p53 has been shown to be activated in several ways: posttranslational modifications of the C terminus, such as acetylation or phosphorylation; association with the monoclonal antibody PAb421; or deletion of the C-terminal domain (Prives, C. & Hall, P. A. 1999 J Pathol 187:112-126).

In contrast to these observations, the experiments described herein demonstrate that umnodified, full-length p53 binds very efficiently to its natural recognition sequences within both DNA and chromatin. This occurs in the absence of p53 modification by acetyl transferases or chromatin disruption by ATP-dependent remodeling complexes. In fact, perturbations of the C-terminal domain reveal the distinct nature of individual p53 binding sites. For example, deletion of the C terminus or association of this domain with PAb421 actually abolishes p53 interaction with the 3′ site within the p21 promoter. Conversely, both modifications result in the switch from latent to active DNA binding by p53 when analyzed by EMSA. The discrepancy between p53 binding results obtained with EMSA using short oligonucleotides and DNase footprinting using larger promoter fragments can be resolved by increasing the length of the oligonucleotides so that it presumably adopts a secondary structure to which p53 can stably bind. Importantly, Cain et al. (2000 J Biol Chem 275:39944-39953) have shown that modifications of the N terminus greatly affect p53 binding to the p21 promoter (5′ site) as measured in footprinting assays. This is especially significant because the N terminus is the site of multiple posttranslational modifications and can associate with critical cofactors such as MDM2, TAFs, and TRAPs (Cain, C. et al. 2000 J Biol Chem 275:39944-39953 and references therein).

Seminal experiments by Kim and colleagues demonstrated that cruciforms or other non-B-DNA structures are normal recognition elements for p53 (Kim, E. et al. 1997 Oncogene 15:857-869; Kim, E. et al. 1999 Oncogene 18:7310-7318). This would explain why unmodified p53 fails to bind to a short DNA oligonucleotide in EMSA, because this sequence cannot readily form the secondary structure it would usually adopt in a larger piece of DNA. Thus, modification by acetylation, antibody association, or deletion of the C terminus might be required for p53 to bind to a DNA structure that the native protein normally does not recognize. This interpretation by Kim et al. (1997, and 1999 supra) is entirely consistent with the results presented herein. More recently, it was demonstrated that p53 can be specifically directed to cruciform structures, even though the sequences forming the cruciform do not fit the p53 consensus binding site (Jett, S. D. et al. 2000 J Mol Biol 299:585-592). Taken together, these studies emphasize the importance of using DNA recognition sequences within an appropriate context when examining the effect of both C- and N-terminal modifications on p53 binding. Moreover, the results may differ depending upon the specific target gene and p53 recognition sequence used.

It is interesting to note that p53 bound to the p21 promoter with higher affinity, and with different kinetics, when assembled into chromatin than it does to DNA. Particularly because this occurs in the absence of chromatin remodeling or modifying complexes and is not observed with other transcription factors that can also bind to nucleosomes, such as Gal4-VP16 (Pazin, M. J. et al. 1998 J Biol Chem 273:34653-34660). This could be explained if bending of the DNA, when wrapped around a nucleosome, generates a secondary structure that is more stable for p53 binding. Indeed, previous studies determined the importance of DNA bending for p53 high-affinity binding and predicted that some p53 binding sites would be exposed and accessible when incorporated into a nucleosome (Nagaich, A. K. et al. 1999 PNAS USA 96:1875-1880). Importantly, these findings demonstrate that the structure recognized by p53 in p21 promoter DNA is preserved and improved or stabilized in chromatin.

The physiological significance of the distinct kinetics of p53 occupancy observed on the p21 promoter as chromatin or DNA is unclear. The linear rate of association of p53 with chromatin may indicate that lower concentrations of p53 are required to fully occupy binding sites in vivo than the cooperative binding to DNA would indicate. This could be significant if the cell has to respond efficiently to activate p53-responsive pathways without waiting for a critical threshold of p53 concentration to be reached. It should be emphasized, however, that the nature of p53 binding to chromatin and the requirements for remodeling/modifying activities may vary with individual target promoters.

One can surmise that the variable nature of DNA consensus sequences recognized by p53 is a critical regulatory feature, because it enables diverse structures to be generated within target promoters and manipulated by a variety of cellular signals. In fact, the ability of p53 to function as an activator or repressor and differentially recruit cofactors may be determined by the conformation it assumes when bound to DNA/chromatin sites having specific topologies. Such allosteric regulation has been demonstrated for the transcription factor, Pit-1, which can switch from an activator to a repressor by a two base pair change in its binding sites (Scully, K. M. et al. 2000 Science 290:1127-1131).

The following examples are illustrative, and should not be construed to limit the present invention.

### EXAMPLE 1

Plasmids and Expression of Recombinant Proteins

The WWP-LUC (p21-LUC) plasmid was constructed as described in El-Deiry et al. (El-Deiry, W. S. et al. 1995 Cancer Res 55:2910-2919). Full-length human Flag-tagged p53 was expressed in Sf9 cells and purified from total cell extracts by affinity to anti-Flag M2 affinity gel (Sigma) according to the protocol used to purify Flag-tagged dACF described by Ito et al. (Ito, T. et al. 1999 Genes & Dev 13:1529-1539). The C-terminal deletion mutant p53AC30 (amino acid 1-353) protein was purified from Sf9 cells as described by Cain et al. (Cain, C. et al. 2000 J Biol Chem 275:39944-39953). cDNA encoding the p53KR was subcloned into baculovirus expression vector (Bac-to-Bac System, Gibco BRL), and the protein was purified by similar means. Recombinant ACF complex (Flag-tagged dACF+untagged dISWI) was produced in Sf9 cells and purified by anti-Flag M2 (Sigma) affinity chromatography as described by Ito et al. (Ito, T. et al. 1999 Genes & Dev 13:1529-1539). Histidine-tagged NAP-1 was purified from baculovirus-infected Sf9 cells by Ni-NTA (Qiagen) affinity chromatography, followed by a conventional Mono-Q chromatographic step. Full-length human histidine-tagged p300 was expressed in Sf9 cells and purified as previously described by Kraus et al. (Kraus, W. L. et al. 1999 Mol Cell Biol 19:8123-8135).

Electrophoretic Mobility Shift Assay

Acetylation of p53 by p300 was performed as described (Gu, W. and Roeder, R. G. 1997 Cell 90:595-606). EMSA was carried out essentially as described (Jayaraman, L. et al. 1998 Genes & Dev 12:462-472). The 25 bp double-stranded oligonucleotide harboring the 5′ site of the p21 promoter was generated by annealing the single-stranded oligonucleotides 5′-CAGGAACATGTCCCAACATGTTGAA-3′ (SEQ ID NO: 5) and 5′-TTCAACATGTTGGGACATGTTCCTG-3′ (SEQ ID NO: 6). The 160 bp region of the p21 promoter containing a centered 5′ site was generated by PCR from the plasmid p21-LUC, using the primers 5′-CGGGCTGCAGGAATTCGATATC-3′ (sense) (SEQ ID NO: 7) and 5′-CCATCCCCTTCCTCACCTGATA-3′ (antisense) (SEQ ID NO: 8). Both probes were ³²P end-labeled using T4 polynucleotide kinase and further purified by 12%-15% polyacrylamide native gels.

Chromatin Assembly and In Vitro Transcription

Chromatin was reconstituted using Drosophila embryonic extracts as described (Bulger, M. and Kadonaga, J. 1994 Methods Mol Genet 5:242-262). After assembly, the chromatin template (500 ng of DNA in 50 μl) was incubated with p53 and/or p300 (typically between 0.2-10 pmol of p53 and 0.2-0.5 pmol of p300), for 30 min at 27° C. For transcription, 20 μl of HeLa cell nuclear extract (typically 8 mg/ml) was added and incubated on ice for 10 min, then reactions proceeded as described (Kadam, S. et al. 2000 Genes & Dev 14:2441-2451). The purified RNA was analyzed by primer extension analysis with primers specific to luciferase and -globin gene (used as an internal control) sequences.

DNase I Footprinting of Chromatin and DNA

DNase I footprinting was done using three different templates: crude chromatin-assembled mixtures, CL4B-purified chromatin, and naked DNA. For purification of chromatin assembled with Drosophila embryonic extracts, methods were used as previously described (Mizuguchi, G. and Wu, C. 1999 in: Peter Becker (Ed.) Chromatin Protocols. Totowa, N.J.: Humana Press). After incubation of the different templates with the proteins, samples were digested with 33 U/ml (crude chromatin), 1.5 U/ml (purified chromatin) or 0.1 U/ml (naked DNA) DNase I (Boehringer Mannheim) for 75 s at 25° C. Purified DNA fragments were analyzed by primer extension.

Western Blot Analysis

For Western blot analysis of chromatin samples, mixtures were boiled for 15 min in SDS-PAGE loading buffer and resolved by 10% SDS-PAGE electrophoresis. After transfer of proteins to PVDF membranes, filters were blocked and incubated with monoclonal antibody DO-1 (unmodified N-terminal domain of p53, Oncogene Research Products), polyclonal antibodies PC198 (acetylated p53, Oncogene Research products), and polyclonal antibodies C20 (p300, Santa Cruz Biotechnology). Blots were developed using Amersham-enhanced chemiluminescence reagents.

Chromatin HAT Assay

Chromatin assembly was performed with recombinant dACF complex and NAP-1 histone chaperone as described (Ito, T. et al. 1999 Genes & Dev 13:1529-1539 and references therein). Chromatin assembly was assessed by micrococcal nuclease digestion and transcriptional repression. After assembly, proteins were added to the chromatin mixtures in the presence of 5 μM ³H-acetyl CoA (ICN). After incubation for 1 hr at 30° C. with p53 and/or p300, reactions were stopped by boiling in SDS-PAGE loading buffer and analyzed on a 15% SDS-PAGE gel. The gel was fixed and treated with fluorography enhancing solutions (NEN Life Science). The gel was then dried and exposed to autoradiography.

Chromatin Immunoprecipitation Assay

Chromatin immunoprecipitation analysis was performed using the anti-acetylated histone H4 antibody (Upstate Biotechnology) according to the manufacturer's recommendations and as previously described (Kundu, T. K. et al. 2000 Mol Cell 6:551-561) with some minor modifications, and as described in Example 2 (see below).

### EXAMPLE 2

Chromatin Immunoprecipitation (ChIP) Protocol

U2OS cells were grown to 50-60% confluency and treated either with UV-C or doxorubicin, (constant presence in media) for various times before crosslinking. After aspirating the media and washing with phosphate-buffered saline (PBS), cells were crosslinked with a 1% formaldehyde solution in 1×PBS for 15 min at room temperature. Crosslinking was stopped by addition of glycine to 125 mM final concentration. Monolayers were washed twice with cold PBS and harvested in radioimmunoprecipitation assay (RIPA) buffer [150 mM NaCl, 1% v/v Nonidet P-40, 0.5% w/v deoxycholate, 0.1% w/v SDS, 50 mM Tris pH 8, 5 mM EDTA, protease inhibitor cocktail, phosphatase inhibitors (20 mM NaF/0.2 mM sodium orthovanadate), deacetylase inhibitors (5 μM trichostatin A/5 mM sodium butyrate) and 0.5 mM PMSF] to generate a suspension of 10 million cells/ml. Suspensions were sonicated to generate DNA fragments below 500 bp and clarified by centrifugation for 10 min at 12000 g. Protein solutions were quantified for protein and DNA content and adjusted to 1 mg/ml of protein.

For immunoprecipitation, 1 mg of protein extract was pre-cleared for 2 hrs with 40 μl of a 50% slurry of 50:50 proteinA:proteinG sepharose before addition of indicated antibodies. Antibody (Ab) sources: DO1 (anti-total p53, Oncogene), S15P (anti-Serine 15-phospho-p53, Cell Signaling), AcH4 (Upstate), anti-TAF250 (R. Tjian), anti-TFIIB, -TBP, -RNApol II, -TFIIH (Santa Cruz), anti-serine 2-phospho-RNApol II and anti-serine 5-phospho-RNApol II (Covance). 5 μl of each Ab were added and incubation took place for 12 hrs at 4° C. in the presence of 40 μl of a 50% slurry of 50:50 proteinA:proteinG sepharose pre-blocked with 1 mg/ml bovine serum albumine (BSA) and 0.3 mg/ml of sonicated salmon sperm DNA. Sepharose beads were recovered by centrifugation for 1 min at 6000 g and washed twice with RIPA buffer, four times with ChIP Wash Buffer [100 mM Tris HCl pH 8.5, 500 mM LiCl, 1% v/v Nonidet P-40, 1% w/v deoxycholic acid], twice with RIPA buffer and twice with 1×TE. Immunocomplexes were eluted for 10 min at 65° C. in the presence of 1% SDS and crosslinking was reversed by adjusting to 200 mM NaCl and incubating 5 hrs at 65° C. Samples were treated with Proteinase K followed by phenol-chloroform extraction.

DNA was precipitated and used as template in semi-quantitative PCR reactions. All reactions were performed in the linear range of amplification which varied from 25 to 32 cycles, according to the primer combination and Ab used. To ensure linearity, control PCR reactions were performed at −/+1 cycle with twice and half the amount of sample. Amplification products were electrophoresed in either 2% agarose gels and visualized by ethidium bromide or in 5% polyacrylamide gels and visualized by autoradiography (³²P-dCTP included in PCR reaction). All products were approximately 200 bp and contained the elements described above. Variable amounts of input DNA were also included as controls. When Abs against phosphorylated C-terminal domain (CTD) were used, immunocomplexes were recovered using protein G-sepharose beads precoated overnight with 2 mg/ml goat-anti mouse IgM (Sigma). Different immunoprecipitations were performed using aliquots of the same protein extracts. PCR reactions using different primer combinations were performed from the same DNA samples.

Immunofluorescence and RNA FISH Protocol

Human fibroblasts were grown to 30-50% confluency on glass coverslips. Cells were stressed as indicated for ChIP experiments. Cells were fixed in 4% paraformaldehyde, washed twice with PBS-2 μg/ml Heparin and incubated with Blocking Solution (1 mg/ml BSA, 5% Swine Normal Serum, %5 Goat Normal Serum, 1% Triton X100, 1×PBS, 20 μg/ml Heparin) for 30 min at room temperature (RT). Cells were incubated with 1:100 Ab dilutions (polyclonal S15P, Cell Signaling or monoclonal anti-p21, Santa Cruz) in Blocking Solution for 1 hr at RT, washed three times in PBS-Heparin, incubated with 1:1000 dilutions of secondary antibody (anti-rabbit or anti-mouse coupled to Alexa 488, Molecular Probes) and washed again. Samples were re-fixed with 2% paraformaldehyde before RNA FISH.

For RNA fiber-fluorescence in situ hybridization (FISH), p21 cDNA was labeled with biotin using Biotin Nick Translation Kit (Roche) and used as probe on samples previously treated to detect either p21 protein or S15P-p53. 100 ng of probe containing 15 μg of tRNA and 20 μg of salmon sperm DNA were used to hybridize cells on coverslips for 12 hrs at 37° C. in Hybridization Solution [50% formamide, 1 mg/ml Blocking Reagent (Roche), 10% w/v Dextran Sulfate, 2×SSC, 20 μg/ml Heparin]. Samples were washed once with 50% formamide at 45° C., once with 2×SSC at 45° C. and three times with 2×SSC at RT. Probe was detected by consecutive incubation with avidin-texas red (Vector Labs, 1:1000 dilution in Blocking Solution), biotinylated-anti-avidin (Vector Labs, 1:500) and avidin-texas red again. DNA was detected by diamidinophenylindole (DAPI) staining and coverslips were mounted on glass slides using a solution of 90% glycerol, 0.21 M DABCO (1,4-diazabicyclo[2.2.2]octane). Cells were visualized using confocal deconvolution microscope (Deltavision).

The size and source of cDNAs used for RNA FISH were as follows:

p21: 2.1 kb from Dr. Wade Harper, Baylor College of Medicine.

Fas/Apol: 2.55 kb from Dr. Peter Krammer, DKFZ, Germany.

PUMA (p53 upregulated modulator of apoptosis): 600 bp from Dr. Bert Vogelstein, Johns Hopkins University.

Primer Sequences:

| | |
|---|---|
| −5 kb amplicon: | |
| sense: | |
| GGATCCCTGTAGAGATGCTCAGGCTGC | (SEQ ID NO: 9) |
| antisense: | |
| CGGCAAATCTGGTTGGCATCATCTCGC | (SEQ ID NO: 10) |
| 5′ site amplicon: | |
| sense: | |
| GCTTTCCACCTTTCACCATTCCCCTACC | (SEQ ID NO: 11) |
| antisense: | |
| GTCTCCTGTCTCCTACCATCCCCTTCC | (SEQ ID NO: 12) |
| 3′ site amplicon: | |
| sense: | |
| GGTCTGCTGCTGTGTCCTCCCACC | (SEQ ID NO: 13) |
| antisense: | |
| CCCACTGAAAAACAGAACCCAGGCTT | (SEQ ID NO: 14) |
| TATA box amplicon: | |
| sense: | |
| TGCTGGAACTCGGCCAGGCTCAGCTG | (SEQ ID NO: 15) |
| antisense: | |
| CCAGCTCCGGCTCCACAAGGAACTG | (SEQ ID NO: 16) |
| +5 kb amplicon: | |
| sense: | |
| TTGGCAGAGCAGGGTTACCCTACTTGG | (SEQ ID NO: 17) |
| antisense: | |
| AGGACCAGGGTCCTGTTTGCCACCAG | (SEQ ID NO: 18) |

*****

Although the invention has been described with reference to embodiments and examples, it should be understood that various modifications can be made by a skilled artisan without departing from the spirit of the invention. Accordingly, the invention is limited only by the following claims. All references cited herein are hereby expressly incorporated by reference.

What is claimed is:

1. A method for identifying compounds that modulate the binding of p53 protein to a gene of interest, comprising:
providing said gene of interest as chromatin-assembled DNA;
contacting said gene of interest with said p53 protein and a test compound; and
determining whether the presence of said test compound modulates the binding of said p53 to said gene of interest.

2. The method of claim 1, wherein said p53 protein comprises a human p53 protein.

3. The method of claim 1, wherein said p53 protein comprises recombinant p53 protein.

4. The method of claim 1, wherein said p53 protein comprises p53 protein that is purified from tissues.

5. The method of claim 1, wherein contacting said gene of interest further comprises contacting said gene of interest with a cell nuclear extract.

6. The method of claim 1, wherein said gene of interest is a cell cycle control gene or an apoptotic gene.

7. The method of claim 1, wherein said p53 protein is a full-length p53 protein.

8. The method of claim 1, further comprising determining whether the presence of said test compound modulates p53 controlled transcription of said gene of interest.

9. The method of claim 1, wherein determining whether the presence of the test compound modulates the binding of p53 comprises a footprinting assay, an electrophoretic mobility shift assay or an chromoimmunoprecipitation assay.

10. The method of claim 1, wherein said test compound is selected from the group consisting of: proteins, peptides, antibodies, small organic molecules, and inorganic molecules.

11. The method of claim 1, wherein determining whether the presence of the test compound modulates the binding of p53 comprises determining whether the presence of the test compound decreases the binding of p53.

12. The method of claim 1, wherein determining whether the presence of the test compound modulates the binding of p53 comprises determining whether the presence of the test compound increases the binding of p53.

13. A method for identifying a modulator that inhibits growth of cancer cells, comprising:
providing a gene construct comprising a chromatin-assembled promoter linked to a reporter gene, wherein said promoter binds p53 protein;
incubating said gene construct in the presence of p53 protein and a test compound; and
determining whether said test compound increases binding of said p53 protein to said gene construct by measuring the amount of reporter gene activity in the presence and the absence of said test compound.

14. The method of claim 13, wherein said promoter comprises the p21 promoter.

15. The method of claim 13, wherein said reporter gene comprises the luciferase gene.

16. The method of claim 13, wherein said modulator is selected from the group consisting of: proteins, peptides, antibodies, small organic molecules, and inorganic molecules.

17. The method of claim 13, wherein said cancer cells are tumor cells.

18. The method of claim 13, wherein said promoter is a pro-apoptotic promoter.

19. A method for identifying a test compound that modulates the interaction of p53 protein with a transcription cofactor that associates with p53 protein when p53 protein is bound to a gene of interest, comprising:
providing said gene of interest as chromatin-assembled DNA;
contacting said gene of interest with said p53 protein, the transcription cofactor, and the test compound; and
determining whether the presence of said test compound modulates the association of said cofactor with said p53 protein bound to said gene.

20. The method of claim 19, wherein said cofactor is purified from an expression system.

21. The method of claim 19, wherein said cofactor is present in a tissue extract.

22. The method of claim 21, wherein said tissue extract is a protein extract.

23. The method of claim 21, wherein said extract is from a tissue affected by a neoplastic, autoimmune, or neurodegenerative disorder.

24. The method of claim 21, wherein said tissue extract is from a cell treated with a DNA damaging agent.

25. The method of claim 19, wherein said gene of interest comprises a pro-apoptotic promoter, a cell cycle promoter or a tissue specific promoter.

26. The method of claim 19, wherein said gene of interest comprises a p21 promoter.

27. The method of claim 19, wherein said transcriptional cofactor is selected from the group consisting of: histone acetyl transferase cofactor p300, Pit-1, MDM2, TAFs, TRAP, the CREB binding protein (CBP) and p300 (CBP/p300), and both CBP/p300 and p300/CBP-associated factor (PCAF).

28. A kit for identifying test compounds that affect p53 protein binding to a promoter, comprising:
a gene construct comprising a chromatin assembled p53 binding site genetically linked to a reporter gene; and
isolated p53 protein.

29. The kit of claim 28, wherein said reporter gene encodes a detectable protein.

30. The kit of claim 29, wherein said detectable protein is an enzyme or a fluorescent protein.

31. The kit of claim 30, wherein said enzyme is luciferase.

32. The kit of claim 30, wherein said fluorescent protein is a green fluorescent protein.

33. The kit of claim 28, further comprising at least one transcriptional cofactor.

34. The kit of claim 33, wherein said transcriptional cofactor is selected from the group consisting of: histone acetyl transferase cofactor p300, Pit-1, MDM2, TAFs, TRAP, the CREB binding protein (CBP) and p300 (CBP/p300), and both CBP/p300 and p300/CBP-associated factor (PCAF).

35. The kit of claim 33, wherein said transcriptional cofactor is isolated from a nuclear extract.


SEQUENCE LISTING (18 sequences)

SEQ ID NO: 1 - DNA, 20 bp, Artificial Sequence
  p21 promoter
agactgggcatgtctgggca

SEQ ID NO: 2 - DNA, 20 bp, Artificial Sequence
  p21 promoter
tctgacccgtacagacccgt

SEQ ID NO: 3 - DNA, 20 bp, Artificial Sequence
  p21 promoter
gaacatgtcccaacatgttg

SEQ ID NO: 4 - DNA, 20 bp, Artificial Sequence
  p21 promoter
cttgtacagggttgtacaac

SEQ ID NO: 5 - DNA, 25 bp, Artificial Sequence
  oligonucleotide
caggaacatgtcccaacatgttgaa

SEQ ID NO: 6 - DNA, 25 bp, Artificial Sequence
  oligonucleotide
ttcaacatgttgggacatgttcctg

SEQ ID NO: 7 - DNA, 22 bp, Artificial Sequence
  primer
cgggctgcaggaattcgatatc

SEQ ID NO: 8 - DNA, 22 bp, Artificial Sequence
  primer
ccatccccttcctcacctgata

SEQ ID NO: 9 - DNA, 27 bp, Artificial Sequence
  sense primer
ggatccctgtagagatgctcaggctgc

SEQ ID NO: 10 - DNA, 27 bp, Artificial Sequence
  antisense primer
cggcaaatctggttggcatcatctcgc

SEQ ID NO: 11 - DNA, 28 bp, Artificial Sequence
  sense primer
gctttccacctttcaccattcccctacc

SEQ ID NO: 12 - DNA, 29 bp, Artificial Sequence
  antisense primer
gtctcctgtctcctaccatccccttcctc

SEQ ID NO: 13 - DNA, 24 bp, Artificial Sequence
  sense primer
ggtctgctgctgtgtcctcccacc

SEQ ID NO: 14 - DNA, 28 bp, Artificial Sequence
  antisense primer
cccactgaaaaacagaacccaggcttgg

SEQ ID NO: 15 - DNA, 26 bp, Artificial Sequence
  sense primer
tgctggaactcggccaggctcagctg

SEQ ID NO: 16 - DNA, 25 bp, Artificial Sequence
  antisense primer
ccagctccggctccacaaggaactg

SEQ ID NO: 17 - DNA, 27 bp, Artificial Sequence
  sense primer
ttggcagagcagggttaccctacttgg

SEQ ID NO: 18 - DNA, 27 bp, Artificial Sequence
  anatisense primer
aggaccagggtcctgtttgccaccagg